(19) United States
(12) Patent Application Publication
(10) Pub. No.: US 2025/0319294 A1
(43) Pub. Date: Oct. 16, 2025

(54) DRUG DELIVERY ASSEMBLY INCLUDING A MICROPROJECTION FOR EXTENDED DRUG DELIVERY

(71) Applicant: Mott Corporation, Farmington, CT (US)

(72) Inventors: Alex Hill, Manchester, CT (US); David Gonthier, Middletown, CT (US)

(21) Appl. No.: 19/177,058

(22) Filed: Apr. 11, 2025

Related U.S. Application Data
(60) Provisional application No. 63/633,356, filed on Apr. 12, 2024.

(51) Int. Cl. A61M 37/00

(52) U.S. Cl. CPC A61M 37/0015; A61M 2037/0023; A61M 2037/003; A61M 2202/0007; A61M 2205/0244; A61M 2207/00

(57) ABSTRACT

A drug delivery assembly having: a housing having an outer wall, the outer wall having an outer surface and an inner surface, wherein the outer wall extends axially from a first end to a second end and defines a compartment configured to store a first drug; an opening in the housing, the opening positioned at the second end of the housing, with the opening in fluid communication with an area that is external to the housing; a first porous membrane positioned at the opening, wherein the outer wall and the first porous member define at least a part of an exterior of the assembly; and microprojections attached to the exterior of the assembly and configured to deliver a second drug to the area, wherein the second drug is the same as or different from the first drug.

### CROSS REFERENCE

This application claims priority to US 63/633,356, filed on Apr. 12, 2024, the entire contents of which is incorporated herein by reference.

### FIELD OF THE DISCLOSURE

The present disclosure relates to drug delivery assemblies for extended drug delivery and/or tunability and systems/methods for utilizing and fabricating the drug delivery assemblies and, more particularly, to single or dual compartment, and a single or dual porous membrane based (e.g., porous zinc membrane based) drug delivery assemblies for extended drug delivery (e.g., via passive diffusion) and/or tunability.

### BACKGROUND OF THE DISCLOSURE

In general, some drug delivery assemblies or the like are known.

An interest exists for improved drug delivery assemblies, and related methods of use.

Opportunities for improvement are addressed and/or overcome by the assemblies, methods, and devices of the present disclosure.

### BRIEF SUMMARY OF THE DISCLOSURE

The present disclosure provides advantageous drug delivery assemblies for extended drug delivery and/or tunability, and improved systems/methods for utilizing and fabricating the drug delivery assemblies. More particularly, the present disclosure provides single or dual compartment, and single or dual porous membrane based (e.g., porous zinc membrane based) drug delivery assemblies for extended drug delivery (e.g., via passive diffusion) and/or tunability.

More specifically, disclosed is drug delivery assembly, including: a housing having an outer wall, the outer wall having an outer surface and an inner surface, wherein the outer wall extends axially from a first end to a second end and defines a compartment configured to store a first drug; an opening in the housing, the opening positioned at the second end of the housing, with the opening in fluid communication with an area that is external to the housing; a first porous membrane positioned at the opening, wherein the outer wall and the first porous member define at least a part of an exterior of the assembly; and microprojections attached to the exterior of the assembly and configured to deliver a second drug to the area, wherein the second drug is the same as or different from the first drug.

In addition to one or more of the above disclosed aspects of the assembly or as an alternative, the outer wall of the housing defines grooves configured to promote adhesion of the assembly to tissue.

In addition to one or more of the above disclosed aspects of the assembly or as an alternative, the first microprojection is organic.

In addition to one or more of the above disclosed aspects of the assembly or as an alternative, the compartment includes the first drug.

In addition to one or more of the above disclosed aspects of the assembly or as an alternative, the first microprojection includes the second drug.

In addition to one or more of the above disclosed aspects of the assembly or as an alternative, the microprojection includes a microprojection head disposed against the exterior of the assembly, wherein the microprojection head is configured to deliver the second drug to surrounding tissue.

In addition to one or more of the above disclosed aspects of the assembly or as an alternative, the microprojection head is configured to dissolve to thereby deliver the second drug.

In addition to one or more of the above disclosed aspects of the assembly or as an alternative, the microprojection includes a micropassage extending at least partially from the microprojection head at the exterior of the assembly toward the compartment.

In addition to one or more of the above disclosed aspects of the assembly or as an alternative, the micropassage includes a microneedle.

In addition to one or more of the above disclosed aspects of the assembly or as an alternative, the micropassage includes a microchannel.

In addition to one or more of the above disclosed aspects of the assembly or as an alternative, the microprojection head is disposed against the outer surface of the outer wall.

In addition to one or more of the above disclosed aspects of the assembly or as an alternative, the outer wall is porous and the micropassage extends from the outer surface of the outer wall partially toward the inner surface of the outer wall.

In addition to one or more of the above disclosed aspects of the assembly or as an alternative, the outer wall is nonporous and the micropassage extends from the outer surface of the outer wall to the inner surface of the outer wall.

In addition to one or more of the above disclosed aspects of the assembly or as an alternative, the microprojection head is disposed against the porous membrane.

In addition to one or more of the above disclosed aspects of the assembly or as an alternative, one or more of the microprojections includes the second drug and another one or more of the microprojections includes a third drug that differs from the second drug.

In addition to one or more of the above disclosed aspects of the assembly or as an alternative, the first end of the housing includes a septum or a second porous membrane.

Further disclosed is a method of attaching microprojections to a housing of an implantable drug delivery assembly, including: filling the microprojections with a drug; applying the microprojections to the implantable drug delivery assembly; and integrating the microprojections with the assembly.

In addition to one or more of the above disclosed aspects of the method or as an alternative, applying the microprojections to the implantable drug delivery assembly includes one or more of: applying the microprojections to the axial outer wall of the housing that is porous or solid; or applying the microprojections to porous media secured to the axial end of the housing.

In addition to one or more of the above disclosed aspects of the method or as an alternative, integrating the microprojections with the assembly includes chemically bonding the microprojections to the assembly.

In addition to one or more of the above disclosed aspects of the method or as an alternative, chemically bonding the microprojections to the assembly includes applying a self-assembled monolayer to the assembly to improve wetting of the microprojections so that the microprojections adhere to, and precipitate on, the assembly.

The above described and other features are exemplified by the following figures and detailed description.

Any combination or permutation of embodiments is envisioned. Additional advantageous features, functions and applications of the disclosed assemblies, methods and devices of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

The following figures are exemplary embodiments wherein the like elements are numbered alike.

Features and aspects of embodiments are described below with reference to the accompanying drawings, in which elements are not necessarily depicted to scale.

Exemplary embodiments are further described with reference to the appended figures. It is to be noted that the various features, steps, and combinations of features/steps described below and illustrated in the figures can be arranged and organized differently to result in embodiments which are still within the scope of the present disclosure. To assist those of ordinary skill in the art in making and using the disclosed assemblies, methods and devices, reference is made to the appended figures, wherein:

FIG. 1 is a side cross-sectional view of an exemplary drug delivery assembly, according to the present disclosure.

FIG. 2 is a modeled delivery profile of the drug delivery assembly of FIG. 1 and for an exemplary drug.

FIG. 3 is a modeled release profile of drug showing percent released over time for an exemplary drug delivery assembly according to the present disclosure.

FIG. 4 is a side cross-sectional view of another exemplary drug delivery assembly, according to the present disclosure.

FIG. 5 is a side view of another exemplary drug delivery assembly, according to the present disclosure.

FIG. 6 is an exploded partial view of the drug delivery assembly of FIG. 5.

FIG. 7 is an exploded side view of the drug delivery assembly of FIG. 5.

FIG. 8 is a side perspective view of the drug delivery assembly of FIG. 5.

FIG. 9 is a cross-sectional side view of another exemplary drug delivery assembly, according to the present disclosure.

FIG. 10 is a side perspective view of another exemplary drug delivery assembly, according to the present disclosure.

FIG. 11 is an exploded side view of the drug delivery assembly of FIG. 10, and showing the bottom port area in a side cross-sectional view.

FIG. 12 is a cross-sectional side view of another exemplary drug delivery assembly, according to the present disclosure.

FIG. 13 is a cross-sectional side view of another exemplary drug delivery assembly, according to the present disclosure, including at least one microprojection.

FIG. 14 is a cross-sectional side view of part of another exemplary drug delivery assembly, according to the present disclosure, including at least one microprojection, with a remainder of the assembly being the same as one or more other embodiments disclosed herein, including but not limited to the embodiment shown in FIG. 13.

FIG. 15 is a flowchart showing a process of attaching the microprojections to the housing.

### DETAILED DESCRIPTION OF THE DISCLOSURE

The exemplary embodiments disclosed herein are illustrative of advantageous drug delivery assemblies, and systems of the present disclosure and methods/techniques thereof. It should be understood, however, that the disclosed embodiments are merely exemplary of the present disclosure, which may be embodied in various forms. Therefore, details disclosed herein with reference to exemplary drug delivery assemblies and associated processes/techniques of assembly and use are not to be interpreted as limiting, but merely as the basis for teaching one skilled in the art how to make and use the advantageous drug delivery assemblies and/or alternative drug delivery assemblies of the present disclosure.

Disclosed herein are advantageous drug delivery assemblies, and related methods of fabrication and use thereof.

The present disclosure provides improved drug delivery assemblies for extended drug delivery (e.g., via passive diffusion) and/or provides tunability, and improved systems/methods for utilizing and fabricating the drug delivery assemblies.

More particularly, the present disclosure provides single or dual compartment, and single or dual porous membrane based (e.g., porous zinc membrane based) drug delivery assemblies for extended drug delivery (e.g., via passive diffusion) and/or to provide tunability, i.e., drug delivery where the release of the drug can be adjusted to achieve a desired regimen.

Referring now to the drawings, like parts are marked throughout the specification and drawings with the same reference numerals, respectively. The figures are not necessarily to scale, and, for example, in certain views the scale of the parts may have been exaggerated for purposes of clarity.

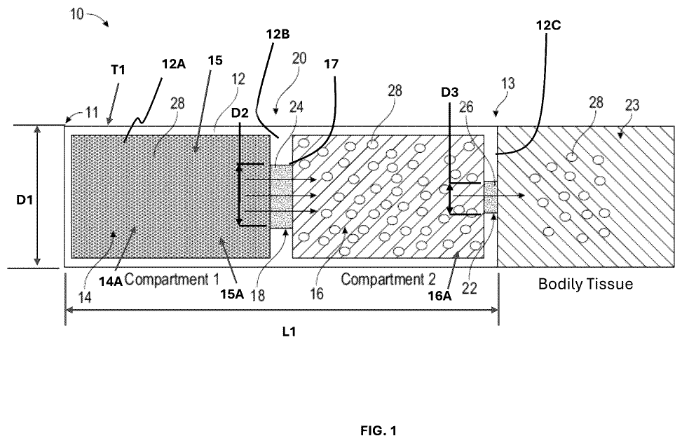

Turning to FIG. 1, there is illustrated a drug delivery assembly 10 depicting an embodiment of the present disclosure.

Exemplary drug delivery assembly 10 takes the form of a dual compartment and dual porous membrane based (e.g., porous zinc membrane based) drug delivery assembly 10 for extended delivery of a first drug 28 (for simplicity, a drug 28), e.g., via passive diffusion, and/or tunability or the like, although the present disclosure is not limited thereto.

As shown in FIG. 1, drug delivery assembly 10 includes a housing 12 that extends from a first end 11 to a second end 13. In exemplary embodiments, the housing 12 is substantially tubular or substantially cylindrical, although the present disclosure is not limited thereto. Rather, it is noted that the housing 12 can take a variety of shapes and/or forms.

The housing 12 can be fabricated from a variety of materials. For example, the housing 12 can be fabricated from a biocompatible metal (e.g., magnesium, zinc, titanium, iron, stainless steel, or an alloy thereof). The housing 12 can also be fabricated from a biocompatible polymer, e.g., a poly (meth) acrylate or copolymer thereof, a polyurethane, a polyether ketone, or the like; or a biodegradable polymer (e.g., a polyester such as a polycaprolactone, polylactide (e.g., poly(DL-lactide (PLA) or poly (L-lactide) (PLLA), or other isomers and copolymers of lactide), a polyglycolic acid, or a copolymer thereof, a polysaccharide, or the like. Where the housing 12 is biodegradable, it is preferably selected to have a slower biodegradability than any biodegradable substance associated with the drug 28, which is described below. In an embodiment, the housing 12 can be fabricated from a material such as zinc, iron, magnesium, titanium, metal alloys, polylactic acid, poly(lactic-co-glycolic acid), or a combination thereof. The outer wall (or outer shell) 12A of the housing 12 can be solid, or can be porous, or a combination of solid and porous, to allow the diffusion of the drug 28 as described below through the housing 12. In an embodiment, all of the outer wall 12A of the housing 12 is solid, except at portions defining the openings, generally referenced as openings 17. In an embodiment, if all or a portion of the outer wall 12A of the housing 12 is porous, it has a porosity that is less than a porosity of the porous membranes as described below.

In non-limiting examples, the housing 12 takes the form of a tube shape, with an overall length of about 0.5 to about 25 cm, preferably about 0.5 to about 10 cm, more preferably between about 1 to about 5 cm. The housing 12 can have a diameter D1 between about 1 to about 25 mm, preferably between about 2 to about 5 mm. The outer wall 12A of the housing 12 can have a wall thickness T1 between about 1 to about 5 mm. It is noted that the diameter D1 of the housing 12 can be between about 3 to about 7 mm, and the length L1 of the housing 12 can be between about 3 to about 12.5 mm.

An exemplary housing 12 defines a first compartment 14 and a second compartment 16 (each generally a compartment 15), with a first opening 18 in the housing 12 in fluid communication with the first and second compartments 14, 16. The first opening 18 is located in a separator wall (or plate) 12B located at a position 20 (e.g., an intermediate position 20) between the first and second ends 11, 13 of the housing 12. The shape of the first opening 18 may be circular in a non-limiting embodiment.

A second opening 22 in the housing 12 is positioned at the second end 13 of the housing 12 of the assembly 10, in an end wall (or plate) 12C, as shown in FIG. 1. The second opening 22 may have a circular cross section with a smaller area than the first opening 18, in a non-limiting embodiment. In general, the second opening 22 can be in fluid communication with an area 23 that is external to the housing 12 of the assembly 10 (e.g., an area 23 such as, for example, the surrounding tissue of a body of a patient, test subject, or the like, after the assembly 10 is positioned in the body). The patient can be a human or animal in need of the drug. The test subject can be a human or animal.

In exemplary embodiments, a first porous membrane 24 is positioned in the first opening 18, and a second porous membrane 26 is positioned in the second opening 22.

The first porous membrane 24 can be attached and/or bonded relative to the first opening 18 of the housing 12 and that the second porous membrane 26 can be attached and/or bonded relative to the second opening 18 of housing 12 via various attachment or bonding methods (e.g., sintering bonding, adhesive, press-fit, etc.).

As indicated above, FIG. 1 shows a cross section of housing 12 of the assembly 10 (e.g., tubular assembly 10), with the assembly 10 having first compartment 14 in fluid communication with second compartment 16 via first porous membrane 24 positioned in first opening 18, and with the assembly 10 having second compartment 16 in fluid communication with an area 23 of the surrounding tissue of a body of a patient via a second porous membrane 26 positioned in the second opening 22.

Each respective interior cavity 14A, 16A (each generally a cavity 15A), i.e., the interior space of the first and/or second compartments 14, 16, can be filled or partially filled with a drug 28, e.g., a solution including the drug 28 in dissolved or particulate form, a gel including the drug 28, a slow release composition including the drug 28, or the like, or a combination thereof. In an embodiment, the drug 28 is provided in dissolved or particulate form in a solution (e.g., water), and fills the compartment 15. When present as particles, the drug can be partially soluble, e.g., in water, and provide extended release of the dissolved drug over time. In other embodiments, the drug 28 is provided in other forms, such as reversibly attached to a gel or solid support (in dissolved or particulate form), present in or attached to a degradable (e.g., dissolvable) matrix such as a gel or solid support, encapsulated in one or more degradable shells, or a combination thereof. Such forms are known to those of skill in the art and are useful to provide even greater tunability of drug release. One or more adjuvants (e.g., salt, pH adjusting agent, or the like) as is known in the art can be present in addition to the drug.

In exemplary embodiments, the first porous membrane 24 is generally less porous, i.e., finer, having a smaller pore size relative to second porous membrane 26, although the present disclosure is not limited thereto. However, it is noted that the mean pore size of the first porous membrane 24 can be as large as about 20 —m, or as large as about 100 —m, or possibly even larger, for example between about 1 to about 500 —m.

The second porous membrane 26 is generally more porous, i.e., coarser, relative to the first porous membrane 24, with the second porous membrane 26 generally having a larger pore size than the first porous membrane 24, although the present disclosure is not limited thereto. An exemplary range of the mean pore size of the second porous membrane 26 can be between about 1 to about 100 μm, or between about 1 to about 500 μm, although the present disclosure is not limited thereto. In some embodiments, a mean pore size of the first porous membrane 24 can be substantially the same as or similar to the mean pore size of the second porous membrane 26.

In exemplary embodiments, the coarseness and microstructure of the second porous membrane 26 can prevent bio-fouling of assembly 10, whereas the fineness of the first porous membrane 24 can allow for precision dosage/control over diffusion/drug 28 delivery to eventual area 23.

As described above, it is also possible for all or a part of the outer wall 12A of the housing 12 to be porous. In an aspect, the outer wall 12A of the housing 12 can be solid at the first compartment 14 and porous at the second compartment 16. Alternatively, the outer wall 12A of the housing 12 can be porous at the first compartment 14 and solid at the second compartment 16. In an embodiment, the outer wall 12A of the housing 12 is fully porous. An exemplary range of the mean pore size of the housing 12 can be between about 1 to about 100μm, or between about 1 to about 500 μm, and can vary, for example being finer in the outer wall 12A of the housing 12 forming the first compartment 14 and coarser in the outer wall 12A of the housing 12 defining the second compartment 16. In some embodiments, a mean pore size of the first porous membrane 24, the second porous membrane 26, and all or a portion of the housing 12 can be substantially the same as or similar to each other. In other exemplary embodiments, the coarseness and microstructure of the second porous membrane 26 and all or a portion of the housing 12 can prevent bio-fouling of assembly 10, whereas the fineness of the first porous membrane 24 can allow for precision dosage/control over diffusion/drug 28 delivery to eventual area 23.

In non-limiting examples, each porous membrane 24, 26 (and optionally all or a portion of the housing 12) that is utilized to regulate the mass transfer of the drug 28 can be fabricated from a biocompatible metal (e.g., magnesium, zinc, titanium, iron, stainless steel, or an alloy thereof). Each porous membrane 24, 26 can also be fabricated from a biocompatible polymer, e.g., a poly (meth) acrylate or copolymer thereof, a polyurethane, a polyether ketone, or the like; or a biodegradable polymer (e.g., a polyester such as a polycaprolactone, polylactide (e.g., PLA or PLLA, etc.), a polyglycolic acid, or a copolymer thereof, a polysaccharide, or the like. Where each porous membrane 24, 26 is biodegradable, each porous membrane 24, 26 is preferably selected to have a slower biodegradability than any biodegradable substance associated with the drug 28 or microprojections, which are described in further detail below. Each porous membrane 24, 26 can be in the shape of a flat cylinder or thin needle, with diameters between about 0.25 to about 10 mm and thicknesses between about 0.25 to about 10 mm, although the present disclosure is not limited thereto.

In general, each compartment 14, 16 is configured and dimensioned to house the drug 28 or active agent particles 28.

As such, FIG. 1 depicts an exemplary drug delivery assembly 10, with the assembly 10 having first and second compartments 14, 16, and having first and second porous membranes 24, 26, configured to regulate the delivery of a drug 28 to area 23 that is external to assembly 10.

The exemplary assembly 10 includes first and second compartments 14, 16 separated by the first porous membrane 24 in a separator plate 12B. The second compartment 16 has two openings 17, that is, a first opening 18 joining the first and second compartments 14, 16, and a second opening 22 that is in communication with area 23.

In exemplary embodiments, the assembly 10 is configured and dimensioned to be implanted in a body of a patient or the like (e.g., within the tissue of a body of a patient or the like). The assembly 10 can be located in other/different areas of the body or the like. More than one assembly 10 can be used, at different locations of the body.

The exemplary assembly 10 is an improvement over other conventional designs/assemblies, as the dual compartment 14, 16 feature of assembly 10 has been shown via modeling to be able to deliver the medication 28 within therapeutic concentrations over longer periods of time to area 23, and to be able to deliver a larger percentage of the loaded dose 28 within those same concentrations. The utilization of slow-or fast-release technologies within the compartment can provide even finer tuning of drug release. For example, a solution including the drug 28 can provide an initial faster (higher concentration) release for a first extended period of time, followed by a slower release over a second extended period of time as the drug is released within the compartment from, for example, a dissolvable gel or encapsulant. The utilization of a plurality of encapsulants, at 12A, for example, can provide multiple release concentrations over multiple extended time periods. For other therapies, where initial slow release (low concentration) is desired for a first extended period of time, followed by faster (higher concentration) over a second extended period of time can be provided, for example, by encapsulating the drug 28 separately at two concentrations, one lower and one higher, or an outer shell having a slower release rate and in inner shell having higher release rate that is exposed after erosion of the outer shell.

Some conventional approaches for extended drug release include an osmotic pump and some polymer matrix based compositions that deliver medication for an extended period of time up to thirty to 120 days (polymer matrix systems), and 180 to 360 days (osmotic pump systems). The passive diffusion based drug delivery of exemplary assembly 10 is able to deliver medication for 180 days or longer, for example, up to 1220 days. Moreover, this exemplary assembly 10 can increase the percent of the initial dose delivered while extending the time spent in a therapeutic concentration window while still maintaining a small footprint.

Furthermore, the second porous membrane 26 separating the second compartment 16 from the external area 23 can be much coarser than the first porous membrane 24 of assembly 10 separating the first and second compartments 14, 16. This can allow for the retention of precise drug delivery with the finer (first) media of membrane 24, and can prevent bio-fouling as the coarser (second) porous membrane 26 is inherently resistant, as determined by testing, to the formation of protein films (e.g., on the second end 13 of the housing 12). In some embodiments, the first porous membrane 24 is coarser (more permeable) than the second porous membrane 26, depending, e.g., on how quickly the drug is intended to reach the patient. A rapid dispensing to a patient may be desired if the drug in the second compartment is not stably stored there, due to the nature of the applied excipient in that compartment. For example, if the drug were stably stored in the first compartment, due to the excipient in that compartment, it may be stored there for an extended period of time, depending on the membrane utilized between the compartments. Then, as the drug diffuses to the second compartment, there may be a desire to have it diffuse to the patient more rapidly because of the different excipient utilized in the second compartment. For that reason, a more permeable membrane may be placed at the outlet of the assembly. It is to be appreciated that various techniques can provide the greater or lesser permeability in the different porous members, and coarseness is one of several nonlimiting options in obtaining the desired permeability.

In an example embodiment, the internal volume of the housing 12 can be about 1 mL, with the internal volume divided into a 0.6 mL compartment defined by the first compartment 14, and with a 0.4 mL compartment defined by the second compartment 16. It is noted that a total internal volume of the first compartment 14 and of the second compartment 16 (added together) can range from about 0.10 mL to 5 mL, preferably between 0.10 to 2 mL.

The overall diameter D1 of the housing 12 of assembly 10 can be about 7 mm, and the length L1 of the housing 12 of the assembly 10 can be about 4 cm. The utilization of extended drug delivery mechanisms as described above, in particular dissolvable gels or a solid support, can result in larger dimensions.

In an example embodiment, the second porous membrane 26 can be classified as a Media Grade (“MG”) 0.1 having a mean pore size of about 0.1 μm. The diameter D3 of this exemplary second porous membrane 26 can be about 0.5 mm by about 1 mm thick. The first porous membrane 24 can be classified as a MG 2, with a diameter D2 of about 1.5 mm by about 3 mm thick. The first and second membranes 24, 26 can be fabricated out of 99.99% pure zinc, although the present disclosure is not limited thereto. It is to be appreciated that the diameter is another variable in controlling the mass transport rate of the drug through the membrane. Thus, the relative sizes of the diameters of the membranes as shown and discussed is not intended on limiting the scope of the embodiments.

In use, exemplary assembly 10 and the other embodiments described herein can be utilized for extended delivery of the drug 28, which includes any therapeutic or active agent such as a medication, pharmaceutical, supplement, or the like, or a reporting compound (e.g., a radiotracer or fluorescent compound) for monitoring drug delivery or other biological process. The drug can be a biologic, a protein, a pharmaceutical or reporter small molecules (10 to 1,000 g/mol), or a combination thereof. In an embodiment, the drug 28 is a biologic. Preferably, the sustained release of the drug 28 is for a period of greater than six months, for example, more than 6 months to two years or more than 6 months to one year. This can overcome difficulties with daily, weekly, or monthly dosing regimens.

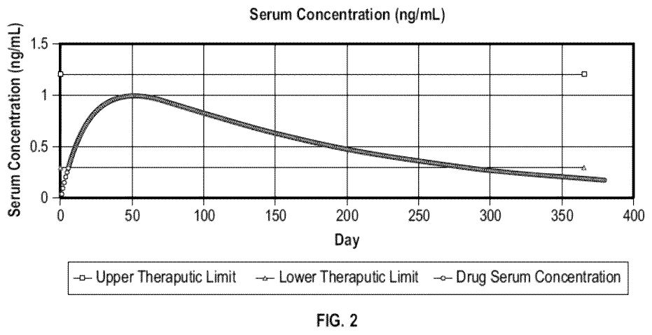

In FIG. 2, the release profile of a drug 28 through an exemplary assembly 10 (FIG. 1) is shown. For this example, the drug 28 was only loaded in the first compartment 14 (behind the first membrane 24), but the drug 28 could be loaded in both compartments 14, 16 theoretically. In FIG. 2, the y-axis details serum concentration, or a description of how concentrated the drug 28 is predicted to be in the blood after x days. The orange and gray lines detail a concentration window where the serum concentration indicates that the drug 28 is effective. The exemplary assembly 10 having both compartments 14, 16 allows for an extension of the time spent in this window of drug effectiveness by better regulating the diffusion gradient between the second compartment 16 and the area 23 by introducing an intermediate volume (compartment 16) and membrane 24 to facilitate mass transfer.

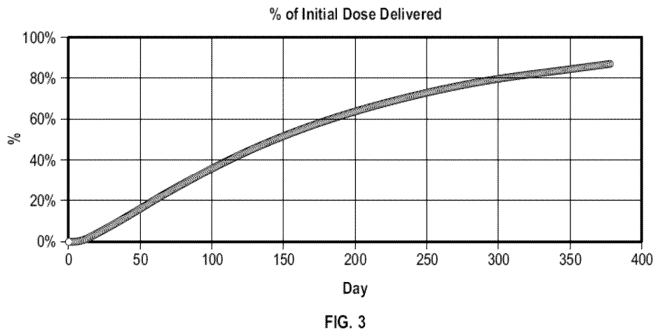

In FIG. 3, the predicted percent released over time relationship for an assembly 10 (FIG. 1) having compartments 14, 16 is shown. A goal of a drug delivery assembly 10 is to have the percent released close to 90% or greater at the time which the serum concentration is modeled to be below the minimum effective therapeutic level. An assembly 10 having both compartments 14, 16, via a better control over the drug delivery physics described above, allows for the achievement of a higher percentage delivered at this time.

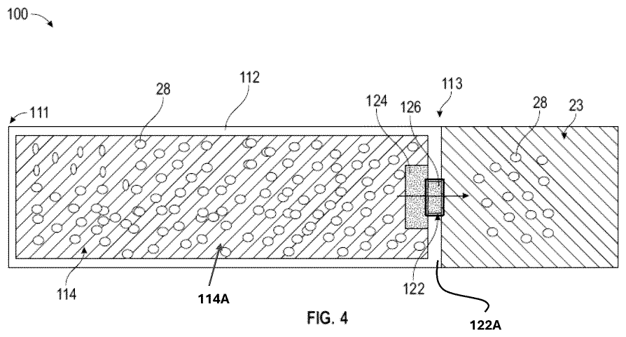

In another embodiment and as shown in FIG. 4, exemplary drug delivery assembly 100 takes the form of a single compartment 114 and dual porous membrane 124, 126 based (e.g., porous zinc membrane based) drug delivery assembly 100 for extended drug 28 delivery (e.g., via passive diffusion) and/or tunability or the like.

The exemplary drug delivery assembly 100 includes a housing 112 that extends from a first end 111 to a second end 113. In exemplary embodiments, the housing 112 is substantially tubular or substantially cylindrical, although the present disclosure is not limited thereto. Rather, it is noted that the housing 112 can take a variety of shapes and/or forms.

The housing 112 can be fabricated from a variety of materials as described above. For example, the housing 112 can be fabricated from a metal (e.g., magnesium, zinc, iron, stainless steel, or an alloy thereof). The housing 112 can also be fabricated from a biodegradable plastic (e.g., PLA or PLLA, etc.), a polyglycolide, a polysaccharide, or the like.

In non-limiting examples, the housing 112 takes the form of a tube shape, as similarly discussed above relative to housing 12 of assembly 10.

The exemplary housing 112 defines a compartment 114. An opening 122 in an end wall 122A of the housing 112 is positioned at the second end 113 of the housing 112 of the assembly 100, as shown in FIG. 4. The opening 122 may have a circular shape in a non-limiting embodiment. In general, the opening 122 can be in fluid communication with an area 23 that is external to the housing 112 of assembly 100 (e.g., an area 23 such as, for example, within the surrounding tissue of a body of a patient, or test subject, after assembly 100 is positioned in the patient).

In exemplary embodiments, a first porous membrane 124 is positioned within the compartment 114, proximal to the opening 122, and a second porous membrane 126 is positioned substantially within the opening 122, as discussed further below.

The first porous membrane 124 can be attached and/or bonded relative to the opening 122 of housing 112 (and/or to membrane 126) and that the second porous membrane 126 can be attached and/or bonded relative to the opening 122 of housing 112 (and/or to membrane 124) via various attachment or bonding methods (e.g., sintering bonding, adhesive, press-fit, etc.).

An interior cavity 114A defined by the compartment 114 can be filled with a drug 28 as described above.

In exemplary embodiments, the first porous membrane 124 is generally finer, having a smaller pore size relative to the second porous membrane 126. In certain embodiments, the first porous membrane 124 has a smaller mean pore size than the second porous membrane 126.

An exemplary range of the mean pore size of the first porous membrane 124 can be between about 0.05 to about 1.0 μm. The second porous membrane 126 is generally coarser relative to the first porous membrane 124, with the second porous membrane 126 generally having a larger pore size than the first porous membrane 124.

An exemplary range of the mean pore size of the second porous membrane 126 can be between about 1 to about 100 μm, although the present disclosure is not limited thereto.

In exemplary embodiments, these two membranes 124, 126, are joined or attached together to form one continuous body, leaving a porous matrix 124 and 126 with a gradient pore size structure. In an embodiment, the two membranes are a single membrane having a porous gradient (stepwise or continuous) of coarser to finer porosities from one surface to the opposite surface.

In exemplary embodiments, the coarseness (larger pores) of the second porous membrane 126 can prevent bio-fouling of assembly 100, and the tightness of the first porous membrane 124 can allow for precision dosage/control over diffusion/drug delivery to the eventual area 23.

In non-limiting examples, each porous membrane 124, 126 utilized to regulate the mass transfer of the drug 28 can be fabricated from zinc, titanium, polyether ether ketone, or another applicable or suitable biomaterial as described for the porous membranes 24, 26. Each porous membrane 124, 126 can be in the shape of a flat cylinder or thin needle, with diameters between about 0.25 to about 5 mm and thicknesses between about 0.25 to about 10 mm, although the present disclosure is not limited thereto.

In general, the compartment 114 is configured and dimensioned to house the drug 28, e.g., whether in solution, as particles, or other form as described above.

As such, FIG. 4 depicts an exemplary drug delivery assembly 100, with the assembly 100 having the compartment 114, and having first and second porous membranes 124, 126, configured to regulate the delivery of the drug 28 to the area 23 that is external to the assembly 100.

In exemplary embodiments, it is noted that the assembly 100 is configured and dimensioned to be implanted in a body of a patient, test subject, or the like (e.g., within the tissue of a body of a patient, test subject, or the like). It is also noted that the assembly 100 can be located in other/different areas of the body or the like, as described above. More than one assembly 100 can be used in different locations of the body.

In use, the exemplary assembly 100 can be utilized for the extended (sustained) delivery of the drug 28. Preferably, the sustained release of the drug 28 is for a period of greater than six months, for example, more than 6 months to two years or more than 6 months to one year. This can overcome difficulties with daily, weekly, or monthly dosing regimens.

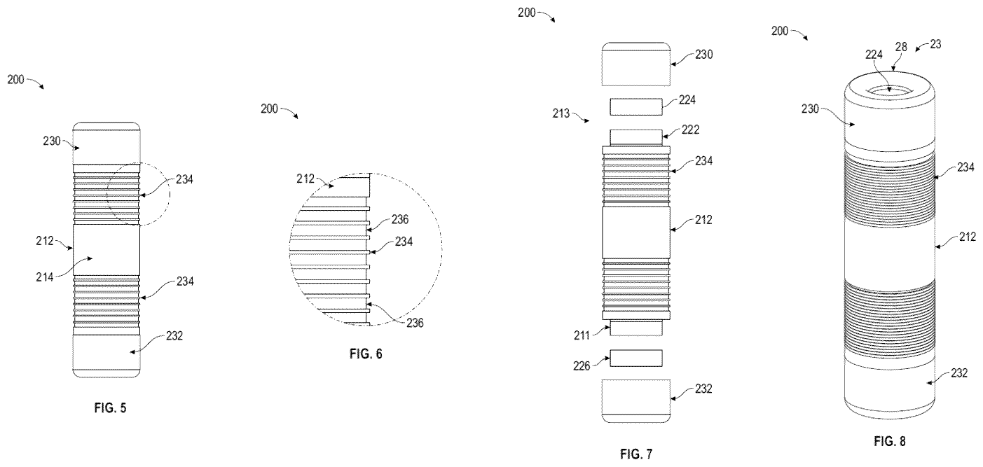

With reference to one or more of FIGS. 5-12, other embodiments of the exemplary drug delivery assembly 200 includes a housing 212 that extends from a first end 211 to a second end 213. In exemplary embodiments, the housing 212 is substantially tubular or substantially cylindrical with a hollow interior, although the present disclosure is not limited thereto. Rather, it is noted that the housing 212 can take a variety of shapes and/or forms.

It is noted that the housing 212 can be fabricated from a variety of materials as described above. For example, the housing 212 can be fabricated from a metal (e.g., magnesium, zinc, titanium, iron, stainless steel, or an alloy thereof). The housing 212 can also be fabricated from a biodegradable polymer (e.g., PLA or PLLA, etc.).

In non-limiting examples, the housing 212 takes the form of a tube or substantially cylindrical shape, as similarly discussed above. In exemplary embodiments, the housing 212 can extend about 1.02 cm or about 1.29 cm or about 1.63 cm from first end 211 to second end 213. In exemplary embodiments, the housing 212 can have a wall thickness of about 0.1016 cm.

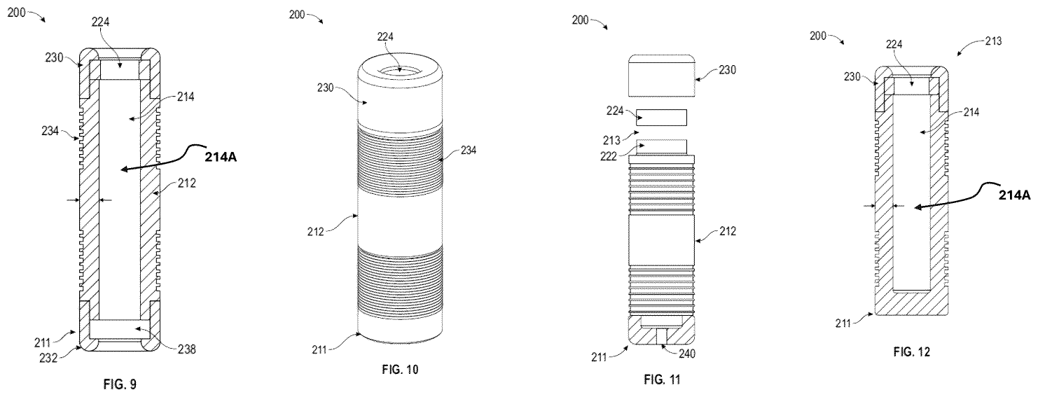

The exemplary housing 212 defines a compartment 214 shown explicitly in FIGS. 9 and 12. The interior cavity 214A of the compartment 214 can be fully or partially filled with a drug 28 as discussed above.

In certain embodiments, a first porous membrane 224 is positioned proximal to the opening 222, and a second porous membrane 226 is positioned proximal to first end 211 as shown in FIG. 7, or distant from first end 211, as discussed further below.

An opening 222 in the housing 212 is positioned at the second end 213 of housing 212 of the implantable drug delivery assembly 200 (otherwise referred to as an assembly or device). In general, opening 222 can be in communication with an area that is external to the housing 212 of the assembly 200 (e.g., an area such as, for example, within the surrounding tissue of a body of a patient or test subject), after the assembly 200 is positioned in the body. In exemplary and non-limiting embodiments, the compartment can have a total internal volume of about 0.25 ml or about 0.50 ml or about 1.00 ml.

In some embodiments, the first porous membrane 224 can be attached and/or secured relative to the opening 222 of the housing 212 via a first end cap 230 attached and/or secured to the second end 213, and the second porous membrane 226 (if adjacent to the first end 211) can be attached and/or secured relative to the first end 211 of the housing 212 via a second end cap 232 attached and/or secured to the first end 211. It is noted that the first end cap 230 can include a hood-like feature that is designed to prevent fibrosis from impeding the diffusion properties of the first porous membrane 224. In exemplary embodiments, the first end cap 230 can have an outer diameter of about 0.34 cm or about 0.43 cm or about 0.54 cm.

An exemplary range of the mean pore size of the first and second porous membranes 224, 226 can each be between about 0.10 to about 100 μm, although the present disclosure is not limited thereto.

In exemplary embodiments, each porous membrane 224, 226 (utilized to regulate the mass transfer of the drug 28) can be fabricated from a material as described above, for example, from zinc, titanium, polyether ether ketone, or another applicable or suitable biomaterial. Each porous membrane 224, 226 can be in the shape of a flat cylinder or thin needle, with diameters between about 0.25 to about 10 mm and thicknesses between about 0.25 to about 10 mm, although the present disclosure is not limited thereto.

Further with reference to FIGS. 5-12, the housing 211 includes at least one grooved or threaded section (or threaded feature) 234 (e.g., two or more sections 234). Such grooved or threaded section can optionally be present in any of the embodiments discussed herein. Each grooved or threaded section 234 includes a plurality of tissue grooves 236 (see, e.g., FIG. 6). As such, the outside surface of the housing 212 is lined with grooved or threaded sections 234 having a plurality of tissue grooves 236, with the tissue grooves 236 promoting the adhesion of tissue to the assembly 200 (e.g., to prevent assembly 200 migration within the body). The grooved or threaded sections 234 modify the surface roughness of the exterior of the assembly 200 to promote tissue growth around the assembly 200 to hold it in place. As such, the grooved or threaded sections 234 along the housing 212 act to promote tissue adhesion and reduce implant assembly 200 migration. In an embodiment, the thread sections 234 conforms to a #6-40 thread classified by ASME B1.1.

Features such as hooks/loops can be added to the housing 212 (or the housing 112) to promote suturing of the assembly 200 (e.g., to a piece of the dermal layer). Additionally, polymeric coatings can be applied to the housing 212 (or the housing 114) to modify the chemical and/or physical properties at the surface of the housing 212 of the assembly 200. It is noted that a large range of surface roughening processes, such as various types of threading, sanding, chemical etching, sand blasting, and other modification methods can also be utilized on the housing 212 (or the housing 112) to promote the adhesion of tissue to the assembly 200. Preferably, raised surface features in the range of 200 to 500 microns can be created on the surface of the housing 212 to promote adhesion of tissue to the assembly 200.

It is noted that an entirely dissolvable design of the housing 112 (or the housing 212) can be fabricated from zinc, and a refillable design of the housing 112 (or the housing 212) can be fabricated from titanium, although the present disclosure is not limited thereto.

FIGS. 5-8 depict an embodiment of an exemplary drug delivery assembly 200, with the assembly 200 defining a first compartment having first and second porous membranes 224, 226 to regulate the delivery of a drug 28 to area 23 that is external to assembly 200.

In an embodiment, the second porous membrane 226 can be attached to a filling apparatus or the like in order to fill the compartment 214 with the drug 28, either before or after the assembly 200 is implanted. The second porous membrane 226 can be closed after filling the compartment 214 with the drug 28 (e.g., closed with a plug of hydroxyapatite cement or other biocompatible cement).

FIG. 9 depicts another exemplary drug delivery assembly 200, with the assembly 200 having the compartment 214, and having first porous membrane 224 secured to housing 212 via first end cap 230, and with a septum member 238 secured to first end 211 via the second end cap 232. The septum member 238 can be attached to a filling apparatus or the like in order to fill compartment 214 with drug 28, either before or after assembly 200 is implanted. The septum member 238 can be closed, if desired, after filling compartment 214 with the drug 28 (e.g., closed with a plug of hydroxyapatite cement or other biocompatible cement, or a solid cap 232 having no opening therein).

FIGS. 10-11 depict an exemplary drug delivery assembly 200, with the assembly 200 having compartment 214, and having first porous membrane 224 secured to the housing 212 via the first end cap 230, and with a drug loading port 240 positioned at the first end 211. The drug loading port 240 can be attached to a filling apparatus or the like in order to fill the compartment 214 with the drug 28 or active agent particles 28, either before or after assembly 200 is implanted. The drug loading port 240 can be closed, if desired, after filling the compartment 214 with the drug 28 (e.g., closed with a hydroxyapatite cement or other biocompatible cement).

FIG. 12 depicts an exemplary drug delivery assembly 200, with the assembly 200 having the compartment 214, and having the first porous membrane 224 secured to the housing 212 via the first end cap 230, and with the first end 211 being closed off. The compartment 214 can be filled with the drug 28 or active agent particles 28 via the second end 213 (e.g., before the membrane 224 is secured to the housing 212).

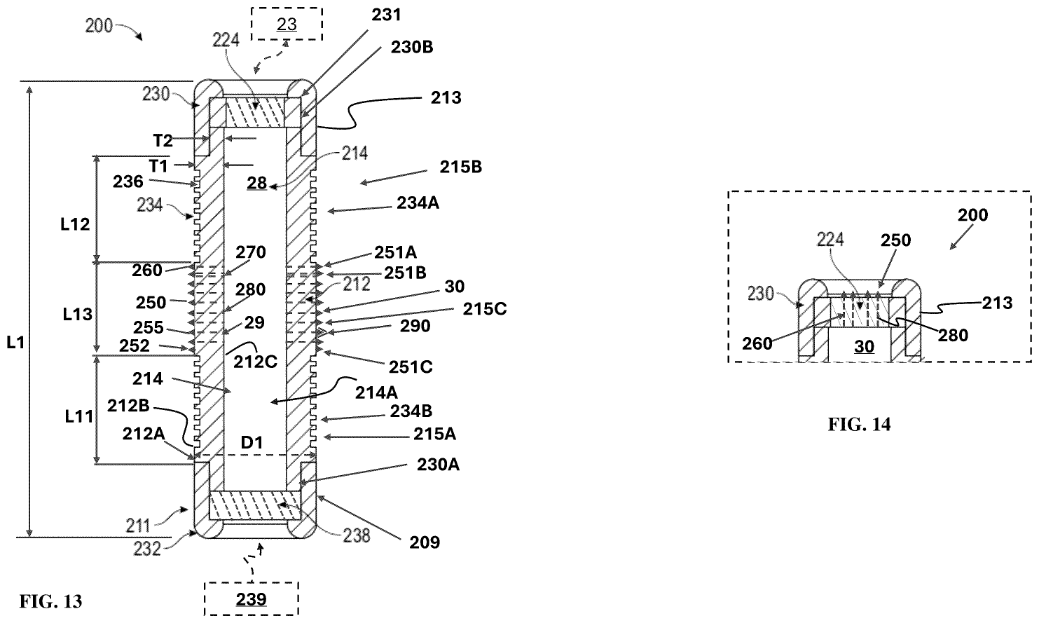

Turning now to FIG. 13, the figure shows another exemplary embodiment of the drug delivery assembly 200. In FIG. 13, reference numbers that are the same as reference numbers in above discussed embodiments may be construed similarly. The assembly 200 has a housing 212 with an outer wall 212A (or shell). The outer wall 212A that may be tubular having a nominal thickness T1 with a diameter D1 and extending between first and second ends 211 by a length L1. The outer wall 212A may be solid (nonporous), porous, or may have axial or circumferential regions that are either solid, porous, or have different porous densities, for targeted delivery of the drug 28, as indicated above. The housing 212 may define the compartment 214 having the interior cavity 214A, and having first porous membrane 224 secured to the second end 213 of the housing 212 via the first end cap 230, and with a septum member 238 secured to the first end 211 of the housing via the second end cap 232. The outer wall 212A, the porous membrane 224, septum member 238 and first and second end caps 230, 232 define the exterior 209 of the assembly 200.

The septum member 238 can be attached to a filling apparatus 239, or the like, in order to fill compartment 214 with the drug 28, either before or after the assembly 200 is implanted. The septum member 238 can be closed, if desired, after filling the compartment 214 with the drug, e.g., closed with a plug of hydroxyapatite cement or other biocompatible cement, or a solid cap 232 that is without an opening. The axial ends 211, 213 of the outer wall 212A of the housing 212 defines first and second cap connecting features 230A, 230B (for simplicity, cap connecting features 231), which may be the same as each other. The cap connecting features 231 are shown as a step decrease in thickness in the outer wall 212A of the housing 212 from a first thickness T1 to a second thickness T2. The cap connecting features 231 may optionally include threads, an adhesive coating, or the like.

The housing 212 may include one or more grooved sections 234 along at least a portion of an outer surface 212B of the outer wall 212A of the house 212. As shown, the housing 211 has two grooved sections, i.e., first and second grooved sections 234A, 234B, respectively disposed at opposite axial ends 211, 213 of the housing 212. That is, along the length L1 of the housing 212, the housing 212 may have first and second axial portions 215A, 215B that are opposite each other and axially inboard of the cap connecting features 231, and an axial center portion 215C. The first and second axial portions 215A, 215B are shown as having the grooves sections 234A, 234B, leaving the axial center portion 252C of the housing 212 without grooves. Though in certain embodiments, the axial center portion 215C has grooves as well. Lengths L11, L12 and L13 of the first, second and center portions 215A-215C of the housing 212 are shown as being the same but that is not intended on limiting the scope of the embodiments.

The first and second grooved sections 234A, 234B may be formed the same as each other, so reference will be to the first grooved section 234A. The first grooved section 234A may be defined by adjacently disposed grooves 236. The grooves 236 may be defined by plurality of full hoop grooves or a continuous helical groove that forms a thread shape. The grooves 236 may function as tissue grooves 236 promoting the adhesion of tissue to the assembly 200, e.g., to prevent assembly 200 migration within the body. The grooved sections 234 result in an increased surface roughness of the outer wall 212A of the assembly 200 to promote tissue growth around the assembly 200 to hold it in place. As such, the grooved sections 234 along the housing 212 act to promote tissue adhesion and reduce implant assembly 200 migration. In an embodiment, where the grooved section 234 forms a thread, the thread may conform to a #6-40 thread classified by ASME B1.1.

According to the embodiments, the center portion 215C includes microprojections 250 (i.e., a plurality of microprojections). The microprojections 250 are adjacent (i.e., attached to or associated with) the external surface 212A of the housing 212. In particular, the assembly 200 has the housing 212 as described above, defining the compartment 214 having the interior cavity 214A, as described above, and a first end 211 and a second end 213 as described above. The second end 213 is in fluid communication with an area 23 outside the housing 212, in the patient in which the assembly 200 is implanted. The first porous membrane 224 as described above is disposed in or on the second end 213. In this embodiment, the septum member 238 is disposed in or on the first end 211, closing it off. The septum member 238 can be attached to the filling apparatus 239 or the like in order to fill or partially fill the interior cavity 214A of the compartment 214 of the housing 212 with the drug 28 as described above, either before or after the assembly 200 is implanted. The septum member 238 can be closed, if desired, after filling compartment 214 with the drug 28, e.g., closed with a plug of hydroxyapatite cement or other biocompatible cement, or a solid cap 232 having no opening therein.

The microprojections 250 can be used in any of the drug delivery assemblies 200 described herein, including the drug delivery assemblies shown in FIG. 1 and in FIGS. 4-12. The septum 238 may or may not be present. The microprojections 250 can be present on any part of the housing 212, including the exterior 212A of the housing 212 defining the first compartment 214A, the second compartment (FIG. 1), or both, whether solid or porous.

In one embodiment, the microprojections 250 are microneedles 260 that are axially adjacent to each other and circumferentially distributed around the hoop-shape of the outer wall 212A and extend from respective microprojection heads 255 at the outer surface 212B of the outer wall 212A toward an inner surface 212C of the outer wall 212A. An annular row 251A of microprojections 250 is shown in FIG. 13, which includes the microneedles 260. It is to be appreciated that all, none or a plurality of the rows of microprojections 250 can be provided as microneedles 260, depending on the application requirements. The microprojection heads 255 can be any suitable shape, for example pyramidal or needle-like, and is discussed in greater detail below.

The depth at which the microneedles 260 extend toward the inner surface 212C may differ based on the porosity of the outer wall 212A. For example, if the outer wall 212 is solid, the microneedles 260 may extend through the thickness of the outer wall 212 to enable access to the drug 28 in the compartment 214. If the outer wall 212 is sufficiently porous in the region of the microneedles 260, then the microneedles 260 may extend only partially through the thickness of the outer wall 212 from the outer surface 212B, being that the drug 28 will be enabled to flow through the wall thickness, from the inner surface 212C to the microneedles 260.

As indicated, the microprojections 250 can be solid or hollow. That is, instead of a microneedle 260 structure with a lumen extending through the thickness of the outer wall 212A, microchannels 270 may be defined, extending from the microprojection head 255 at the outer surface 212B, and through the thickness TI of the outer wall 212A in the locations of the microprojections 250. An annular row 251B of microprojections 250 is shown in FIG. 13, which includes the microchannels 270. It is to be appreciated that all, none or a plurality of the rows of microprojections 250 can be provided as microchannels 270, depending on the application requirements. For simplicity, the microneedles 260 and microchannels 270 may be referred to as micropassages 280. The microprojection heads 255 may define an opening or may be porous to enable a drug within the micropassages 280 to exit when the assembly 200 is implanted.

Each of the micropassages 280 can be loaded with a second drug 29, compared with the first drug 28 in the compartment 214. For example, prior to implanting the assembly 200, and relying on capillary action, a second drug 29 may be first loaded into the compartment 214 and may fill the micropassages 280. Then the first drug 28 may be loaded into the compartment 214. The capillary action would prevent the second drug 29 from flowing out of the microprojection heads 255 while the assembly 200 is outside of the skin of a patient. Once implanted, the second drug 29 would flow relatively quickly out of the microprojections 250 into the patient. This could be beneficial if the second drug 29 was intended on providing a quick infusion of, e.g., a fast-acting medication, ahead of the slower release of the second drug 28 through the porous membrane 224.

Thus, as described above in connection with a first drug 28 present in the one or more compartments 214 of the drug delivery assembly 200, the second drug 29 can be the same or different as the first drug 28 present in the compartment 214. The utilization of the same drug 28 in the compartment 214 and the microprojections 250 allows fine-tuning of the rate of drug delivery. Alternatively, utilizing appropriate manufacturing techniques, one or more of the microprojection 250 can be loaded with a different drug relative to each other, which as indicated would remain within the micropassages 280 prior to implant due to capillary action. For example, the compartment 214 can contain the first drug 28, a portion of the micropassages 280 can be loaded with the same drug as the first drug 28, or the second drug 29 that is different from the first drug 28 (the first and second drugs 28, 29 may be generally referenced as a drug 30). Further, one or more additional drugs, different from the first and second drugs 28, 29, can be loaded within one or more other portions of the micropassages 280. In these embodiment, the drug delivery assembly 200 can be used to deliver two, three, four, or more different drugs.

A combination of solid and hollow microprojections 250 can be used. An annular row 251C of solid microprojections 252 is shown in FIG. 13, which excludes the micropassages 280. It is to be appreciated that all, none or a plurality of the rows of microprojections 250 can be provided as solid microprjections 252, e.g., without micropassages 280, depending on the application requirements. A general reference to microprojections 250 herein includes all configurations of the disclosed microprojections.

The solid microprojections 250 can include the drug 30 as a coating 290 or the drug 30 may be incorporated into the composition of the microprojection head 255. The coating 290 or composition may include a fast-release formulation of the second drug 29, an extended-release formulation of the second drug 29 as described above, or a combination thereof. The fast-release formulation of the second drug 29 can include excipients known in the art for fast dissolution in the body, for example organic materials such as water-soluble polymers, including but not limited to gelatin, dextran, alginate, maltodextrin, a polysaccharide, or a combination thereof; a disintegrant such as polyvinyl pyrrolidone, microcrystalline cellulose, crospovidone, polyethylene oxide, or a combination thereof; or a sugar such as lactose, sucrose, mannitol, or a combination thereof. A combination of a water-soluble polymer and/or disintegrant and/or a sugar can be used in various amounts to tune the drug release rate. Additionally, or alternatively, porosity of the coating or the microprojections 250 can be varied to tune the release rate of the drug from the microprojections 250. Exemplary compositions are described, for example, in U.S. Pat. No. 11,419,816 and U.S. Patent Publication No. 2009/0136554, the disclosure of each of which are incorporated herein by reference in their entirety.

The number and/or size and/or distribution pattern of the microprojections 250 around the outer surface 212A of the housing 212 may also be used to tune the release rate of the drug 30, and the duration of release. In an embodiment, the microprojection heads 255 have a height above, e.g., extending from, the external surface 212A of about 0.1 micrometer to less than about 1 mm, or about 1 micrometer to less than 0.5 mm, or about 1 micrometer to less than about 0.250 mm, or about 1 micrometer to less than about 0.1 mm, or about 1 micrometer to less than about 0.05 mm, or about 1 micrometer to less than about 0.01 mm. All or a part of the external surface 212A of the housing 212 can include the microprojections 250.

In an embodiment, the composition of the microprojection heads 255 is selected to dissolve, e.g., rapidly, over time to deliver a drug 30 to surrounding tissue upon or soon after implantation. The number of microprojections 250 and release rate of the drug 30 from the microprojections 250 can be adjusted to saturate the local area with the drug 30. This may avoid a time period to reach a therapeutically efficacious level of delivery, which can be delayed when using a passive diffusion drug delivery assembly. Accordingly, the microprojections 250 can reduce a time to achieve a therapeutic level of the drug as compared to a drug 30 delivered solely via passive diffusion. In another embodiment, the microprojections 250 can provide an increased strength of an initial dose of a drug 30, followed by delivery at a desired rate from the compartment of the assembly.

As stated above, the microprojections 250 can be hollow, e.g., include a micropassage 280, e.g., wherein the drug 30 is disposed within a microneedle 260. In this embodiment, the drug 30 can be present as a solution, a suspension, or a rapidly-dissolving solid. Materials suitable for utilization as microneedles 260 include the biocompatible materials described above. In an embodiment, the microneedles 260 include an entirely dissolvable material such as zinc or a dissolvable biopolymer, or a metal such as titanium. Implants including microneedles are disclosed, for example, in U.S. Patent Publication Nos. 2018/0296816 and 2011/0046557, the disclosures of each of which is incorporated herein by reference in its entirety. Patches including microneedles are commercially available from Trelleborg AB, headquartered in Trelleborg, Sweden, for example, but these microneedles and patches are limited to subcutaneous delivery.

The microneedles 260 can be of any suitable shape, and can be dimensioned as described above. Selection of the appropriate number and size (e.g., capacity) of the microneedles 260 on an assembly 200 as described herein can be used to adjust delivery of the drug 30, for example, to saturate the local area 23 with the drug 30 upon implantation. This may avoid a time period to reach a therapeutically efficacious level of delivery, which can be delayed when using a passive diffusion drug delivery assembly. Accordingly, the microprojections 250 can reduce a time to achieve a therapeutic level of the drug 30 as compared to a drug delivered solely via passive diffusion. In another embodiment, the microprojections 250 with micropassages 280 can provide an increased strength of an initial dose of a drug 30, followed by delivery at a desired rate from the compartment of the assembly 200.

A process to attach the microprojections 250 to the outer surface 212B of the housing 212 (for example, when the housing outer wall 212A is solid) can include liquid based processing. In such processing, the microprojections 250 are loaded with the drug 30 utilizing appropriate solvent(s) and fractionation, and then applied to the assembly 200. Integration of such microprojections 250 with the assembly 200 is facilitated by appropriate surface chemistry that creates secondary bonding to adhere the microprojections 250 to the assembly 200. For example, integration is obtained by applying a self-assembled monolayer to the assembly 200 to improve wetting characteristics of the microprojections 250 to allow the microprojections 250 to adhere and then precipitate out on the assembly 200. An adhesive also can be used.

Turning to FIG. 14, microprojections 250 with micropassages 280 such as microneedles 260 as a nonlimiting embodiment may be embedded at least partially into the porous membrane 224 that is secured to the second end 213 of the assembly 200 via the first end cap 230. Placement of the microprojections 250 within the porous membrane 224 allows at least a portion of the drug 30 to diffuse through less, i.e., a thinner section, of the porous membrane 224 before entering the microprojections 250 and being more directly diffused into the surrounding tissue. The partial embedding of microprojections 250 can have an effect of increasing the rate of transport of at least a portion of the drug 30 from the assembly 200. In other words, partially embedding one or more of the microprojections 250 in the porous membrane 224 can act to ‘short-circuit’ or partially diminish the filtration function of the membrane 224, on order to maintain a longer period of high-to-moderate release rate of the drug 30.

It is to be understood that the microprojections 250 in the porous media 224 can be used in any of the drug delivery assemblies described herein, including the drug delivery assemblies shown in FIG. 1 and FIGS. 4-13. A septum 238 may or may not be present. Thus, the microprojections 250, with the micropassages 280, such as microneedles 270 as a nonlimiting example, can be present on any part of the housing 12, including the exterior surface 212B of the housing 212 defining the first compartment 214, the second compartment (FIG. 1), or both, whether the outer wall 212A is solid or porous, or within the porous membrane 224.

As described above, the compartment 214 and the microprojections 250 can be loaded with the same or different drugs 30, allowing fine-tuning of release rate and/or the ability to deliver various different drugs. For example a drug delivery assembly 200 can include a compartment 214 filled with a first drug 28, one or more of the microprojections 250 including the same drug 28 to effect a high-release-rate period initially, and further one or more microprojections 250 that access the drug 30 within the assembly 200 after diffusion through only a portion of the thickness T1 of the membrane 224. Such configuration may effect a moderate-to-high release rate period of the drug 30 that extends and tapers the initial high-release rate period.

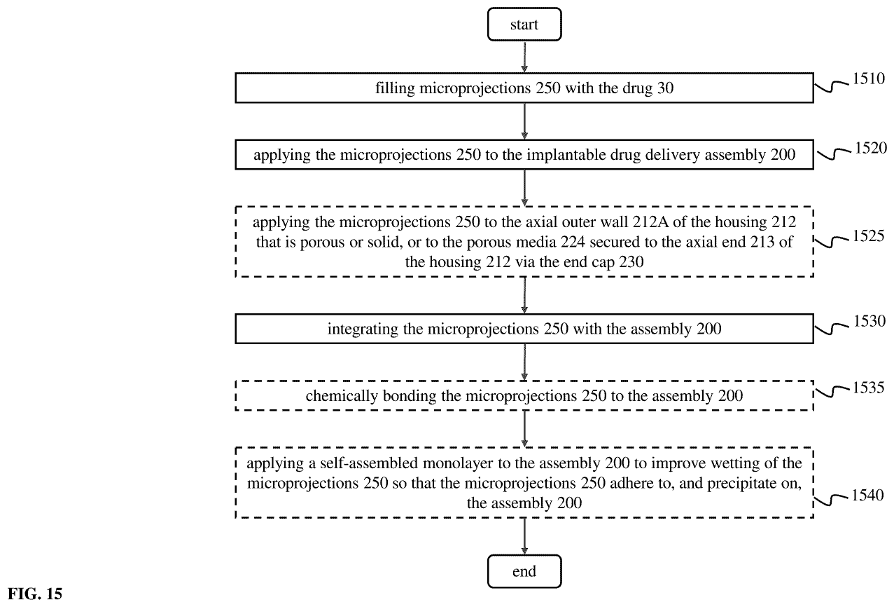

Turning to FIG. 15, a flowchart shows a process to attaching the microprojections 250 to the the housing 212 of implantable drug delivery assembly 200. In FIG. 15, boxes in dashed lines in the flowchart represent further explanations, including alternative embodiments, of one or more preceding steps and are not intended to limit the scope of the embodiments. As shown in block 1510, the method includes filling the microprojections 250 with the drug 30, e.g., using appropriate solvent(s) and fractionation. As shown in block 1520 the method includes applying the microprojections 250 to the drug delivery assembly 200. As shown in block 1525, applying the microprojections 250 to the drug delivery assembly 200 (block 1520) includes applying the microprojections 250 to the axial outer wall 212A of the housing 212 that is porous or solid, or to the porous media 224 secured to the axial end 213 of the housing 212 via the end cap 230. As shown in block 1530 the method includes integrating the microprojections 250 with the assembly 200. As shown in block 1535, integrating the microprojections 250 with the assembly 200 (block 1530) includes chemically bonding the microprojections 250 to the assembly 200, e.g., by applying surface chemistry that creates secondary bonding to adhere the microprojections 250 to the assembly 200. As shown in block 1540, chemically bonding the microprojections 250 to the assembly 200 (block 1535) includes applying a self-assembled monolayer to the assembly 200 to improve wetting of the microprojections 250 so that the microprojections 250 adhere to the assembly 200 and precipitate on the assembly 200.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. “About” as used herein means the usual error range for measurement of the respective value readily known to the skilled person in this technical field, for example ±1%, or ±2%, or ±5% of the stated value. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless defined otherwise, technical and scientific terms used herein have the same meaning as is commonly understood by one of skill in the art to which this application belongs. All patents, patent applications, and other references identified herein are incorporated herein by reference in their entirety. However, if a term in the present application contradicts or conflicts with a term in the incorporated reference, the term from the present application takes precedence over the conflicting term from the incorporated reference.

Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is to be appropriated that the appended claims be construed broadly and in a manner consistent with the scope of the disclosure.

What is claimed is:

1. A drug delivery assembly comprising:
a housing having an outer wall, the outer wall having an outer surface and an inner surface, wherein the outer wall extends axially from a first end to a second end and defines a compartment configured to store a first drug;
an opening in the housing, the opening positioned at the second end of the housing, with the opening in fluid communication with an area that is external to the housing;
a first porous membrane positioned at the opening, wherein the outer wall and the first porous member define at least a part of an exterior of the assembly; and
microprojections attached to the exterior of the assembly and configured to deliver a second drug to the area, wherein the second drug is the same as or different from the first drug.

2. The drug delivery assembly of claim 1, wherein the outer wall of the housing defines grooves configured to promote adhesion of the assembly to tissue.

3. The drug delivery assembly of claim 1, wherein the first microprojection is organic.

4. The drug delivery assembly of claim 1, wherein the compartment includes the first drug.

5. The drug delivery assembly of claim 1, wherein the first microprojection includes the second drug.

6. The drug delivery assembly of claim 1, wherein
the microprojection comprises a microprojection head disposed against the exterior of the assembly, wherein the microprojection head is configured to deliver the second drug to surrounding tissue.

7. The drug delivery assembly of claim 6, wherein
the microprojection head is configured to dissolve to thereby deliver the second drug.

8. The drug delivery assembly of claim 1, wherein
the microprojection comprises a micropassage extending at least partially from the microprojection head at the exterior of the assembly toward the compartment.

9. The drug delivery assembly of claim 8, wherein the micropassage comprises a microneedle.

10. The drug delivery assembly of claim 8, wherein the micropassage comprises a microchannel.

11. The drug delivery assembly of claim 6, wherein
the microprojection head is disposed against the outer surface of the outer wall.

12. The drug delivery assembly of claim 8, wherein
the outer wall is porous and the micropassage extends from the outer surface of the outer wall partially toward the inner surface of the outer wall.

13. The drug delivery assembly of claim 8, wherein
the outer wall is nonporous and the micropassage extends from the outer surface of the outer wall to the inner surface of the outer wall.

14. The drug delivery assembly of claim 6, wherein
the microprojection head is disposed against the porous membrane.

15. The drug delivery assembly of claim 1, wherein
one or more of the microprojections includes the second drug and another one or more of the microprojections includes a third drug that differs from the second drug.

16. The drug delivery assembly of claim 1, wherein
the first end of the housing comprises a septum or a second porous membrane.

17. A method of attaching microprojections to a housing of an implantable drug delivery assembly, comprising:
filling the microprojections with a drug;
applying the microprojections to the implantable drug delivery assembly; and
integrating the microprojections with the assembly.

18. The method of claim 17, wherein
applying the microprojections to the implantable drug delivery assembly includes one or more of: applying the microprojections to the axial outer wall of the housing that is porous or solid; or applying the microprojections to porous media secured to the axial end of the housing.

19. The method of claim 17, wherein integrating the microprojections with the assembly includes chemically bonding the microprojections to the assembly.

20. The method of claim 19, wherein chemically bonding the microprojections to the assembly includes applying a self-assembled monolayer to the assembly to improve wetting of the microprojections so that the microprojections adhere to, and precipitate on, the assembly.
